(19) United States
(12) Patent Application Publication
(10) Pub. No.: US 2016/0090567 A1
(43) Pub. Date: Mar. 31, 2016

(54) Energetic Beer, Flavored Beer or Energetic Drink Based on Beer

(71) Applicants: Martina DITTMAR, Lukavice (CZ); Pavel BEDNÁR, Nové Mesto nad Metuji (CZ); POWER DRINKS SE, Praha 8 (CZ)

(72) Inventors: Martina Dittmar, Lukavice (CZ); Pavel Bednar, Nove Mesto nad Metuji (CZ)

(73) Assignee: Power Drinks SE, Praha 8 (CZ)

(21) Appl. No.: 14/891,144

(22) PCT Filed: May 14, 2014
(86) PCT No.: PCT/CZ2014/000055

§ 371 Date: Nov. 13, 2015

(30) Foreign Application Priority Data
May 15, 2013 (CZ) PUV 2013-27924

(51) Int. Cl. C12C 5/02; A61K 31/145; A61K 31/525; A61K 31/455; A61K 31/197; A61K 31/675; A23L 2/52; A61K 31/522

(52) U.S. Cl. CPC C12C 5/023; C12C 5/02; A23L 2/52; A61K 31/145; A61K 31/522; A61K 31/455; A61K 31/197; A61K 31/675; A61K 31/525

(57) ABSTRACT

Energetic beer, flavored beer or energetic drink based on beer containing 10 to 99.99% of weight of beer, non-alcoholic beer, or a drink based on beer or non-alcoholic beer and it further contains stimulating (energetic or vitamin) substances in the amount of 0.001 to 90% of weight.

### TECHNICAL FIELD

Technical solution relates to a beer or a drink based on beer, especially beer with stimulating (energetic and vitamin) substances and effect.

### BACKGROUND ART

There are known beers or beer-based beverages, which have mostly fruit flavor, exotic fruit flavor, a flavor of mixture of different fruits or spices, cola, honey, etc. Such drink is usually given a flavor by use of specific additives such as the fruit and has a flavor of beer, which is used as the base component.

There are mixed beverages based on alcoholic or non-alcoholic beer and flavored with another alcohol such as tequila.

Object of the invention is to develop a product made of beer that will taste as beer while having stimulating (energetic, vitamin) properties, and which is not known yet on the market and is not present thereon.

### DISCLOSURE OF THE INVENTION

According to this technical solution the energetic beer, flavored beer or energetic drink based on beer has been developed, based on that it contains 10 to 99.99% of weight of beer, non-alcoholic beer, or a drink based on beer or non-alcoholic beer and it further contains stimulating (energetic or vitamin) substances in the amount of 0.001 to 90% of weight.

The proportionate part of the drink with stimulating substances may contain taurine in the amount of 0.01-99% of weight, caffeine in the amount of 0.001-99% of weight, niacin in the amount of 0.0001-50% of weight, pantothenic acid in the amount of 0.00001 to 30% of weight, vitamins B2, B6 and B12 in the amount of 0.0-30% of weight.

### EXAMPLE OF EXECUTION

### Example 1

Energetic non-alcoholic beer 500 ml, was made by mixing 99.75% of weight of light non-alcoholic beer (in the composition: drinking water, barley malt, hops products) and 0.25% of weight of stimulating (energetic and vitamin) substances (in the composition: taurine, caffeine, niacin, pantothenic acid, vitamin B6, vitamin B2).

### Example 2

Energetic beer drink with reduced alcohol content 500 ml, prepared by mixing 25% of weight of light beer-lager (in the composition: drinking water, barley malt, hops products), 5.75% of weight flavoring syrup with lemon flavor, 0.25% of weight of stimulating (energetic and vitamin) substances (in the composition: taurine, caffeine, niacin, pantothenic acid, vitamin B6, vitamin B2) and 69% of weight of carbonated water.

1. Energetic beer, flavored beer or energetic drink based on beer, characterized in that it contains 10 to 99.99% of weight of beer, non-alcoholic beer, or a drink based on beer or non-alcoholic beer and it further contains stimulating (energetic or vitamin) substances in the amount of 0.001 to 90% of weight.
